(19) United States
(12) Patent Application Publication
(10) Pub. No.: US 2014/0271670 A1
(43) Pub. Date: Sep. 18, 2014

(54) COMPOSITIONS FOR TREATING SKIN DISORDERS

(71) Applicant: UNIVERSITY MEDICAL PHARMACEUTICALS, Irvine, CA (US)

(72) Inventors: Raymond Francis, Laguna Beach, CA (US); Gregory Hunt, Corona Del Mar, CA (US)

(73) Assignee: University Medical Pharmaceuticals, Irvine, CA (US)

(21) Appl. No.: 13/834,693

(22) Filed: Mar. 15, 2013

(51) Int. Cl. A61K 39/40; A61K 31/60

(52) U.S. Cl. CPC A61K 39/40; A61K 31/60
USPC 424/164.1; 514/159

(57) ABSTRACT

Compositions and methods for treating skin disorders including acne vulgaris are disclosed. The composition comprises a neutralizing agent directed against the organism and derivatives associated with the skin disorder, a synergistic agent selected from the group consisting of anti-acne actives, anti-microbial actives, antifungal actives, anti-inflammatory actives, exfoliating agents and mixtures thereof; and a pharmaceutically acceptable carrier.

### CROSS-REFERENCE TO RELATED APPLICATION

Not Applicable

### FEDERALLY SPONSORED RESEARCH OR DEVELOPMENT

Not Applicable

### BACKGROUND

This disclosure relates generally to compositions and methods for treating skin-disorders, such as acne. In particular, this disclosure relates to compositions comprising a neutralizing agent directed against the organism associated with the skin disorders and one or more synergistic agents.

Acne is a common skin disorder that is estimated to affect 80-90% of the population at one time in their life. Acne is characterized by comedones, papules, pustules in mild cases, and by nodules and cysts in more severe cases. Areas of the skin with a high sebaceous gland concentration which causes the skin to be oily, such as the face, the upper chest and the back, are most commonly prone to acne. Acne is more common in men than in women during adolescence, but is more common in women during adulthood. Acne can also affect infants. Acne vulgaris, the most common type of acne, is caused primarily by Propionibacterium acnes (P. acnes).

Acne is believed to start with increased production of hormones such as androgens. Young adults going through puberty, for instance, tend to produce more androgens. It is believed that androgens stimulate the sebaceous glands to produce more sebum, which is an oily substance released on the skin.

The high activity of the sebaceous glands results in a local increase in sebum concentration, which agglomerates and forms solid plugs known as comedones in the hair follicles. Formation of comedones in turn causes layers of dead skin, known as keratin, to accumulate, which subsequently may block the hair follicles.

Elevated production of sebum by hyperactive sebaceous glands and blockage of the follicles stimulate the growth of indigenous bacteria living in the follicles, such as P. acnes and Propionibacterium granulosum (P. granulosum).

P. acnes and P. granulosum are relatively slow-growing, non-spore forming, typically anaerobic, Gram positive bacteria which are part of the normal flora present on most healthy adult humans' skin. P. acnes density is much more predominant than that of P. granulosum. P. acnes bacteria are usually just barely detectable on the skin of healthy preadolescents, but their concentration increases significantly during puberty. P. acnes use sebum, cellular debris and metabolic byproducts from the surrounding skin tissue as their primary sources of energy and nutrients. The cellular damage, metabolic byproducts and bacterial debris produced by the rapid growth of P. acnes in follicles can trigger inflammation, which leads to the formation of inflammatory lesions including comedones, pustules, nodules and cysts. Acne often involves physical pain and can lead to scarring on prominent areas of the body, such as the face.

Acne treatments include topical and systematic therapies. Unfortunately, some of these treatments can produce adverse side effects.

Topical treatments include keratolytic agents such as tretinoin and benzoyl peroxide. These agents help clear blocked hair follicles by breaking down keratin. Some of these agents, however, can irritate the skin, causing excessive dryness, scaling, swelling, burning, peeling, redness or allergic reactions. Topical antibiotics, such as clindamycin and erythromycin, can be applied to affected areas to kill P. acnes. However, topical antibiotics can lose their effectiveness if used for a prolonged period of time due to resistance developed by the bacteria.

Systematic antibiotics, often tetracycline or isotretinoin, can be prescribed to manage severe cases of acne. However, antibiotics, when taken orally, may cause many undesirable side effects including abdominal cramps, fatigues, nausea and other various effects. For example, tetracycline increases the sensitivity of skin to sunlight and can prematurely stain the teeth of younger patients. Isotretinoin can cause birth defects.

Hormones such as estrogens and androgens have also been used to treat acne in severe cases. These hormones, when taken orally, however, can increase the risk of blood clots and cancers.

Thus, there is a need for an effective and safe composition for treating and preventing acne. There is also a need for a composition that effectively neutralizes the organism that causes the inflammation associated with acne.

### SUMMARY

In accordance with an aspect of the present disclosure, a pharmaceutical composition for treating and preventing skin disorders comprises: a neutralizing agent directed against at least one organism associated with the skin disorders; a synergistic agent selected from the group consisting of anti-acne actives, anti-microbial actives, antifungal actives, anti-inflammatory actives, exfoliating agents and mixtures thereof; and a pharmaceutically acceptable carrier.

In another aspect, the present disclosure provides a pharmaceutical composition for treating and preventing a skin disorder of bacterial origin, the pharmaceutical composition comprises a neutralizing agent directed against the bacteria associated with the skin disorder; a synergistic agent selected from the group consisting of anti-acne actives, anti-microbial actives, antifungal actives, anti-inflammatory actives, exfoliating agents and mixtures thereof; and a pharmaceutically acceptable carrier. In one embodiment, the skin disorder of bacterial origin is acne vulgaris. In one embodiment, the neutralizing agent comprises an antibody against Propiobacterium Acnes. In one embodiment, the synergistic agent comprises an anti-acne active. In another embodiment, the synergistic agent comprises an exfoliating agent. In one example, the synergistic agent comprises salicylic acid and/or salicylic acid derivatives.

In another aspect of the present disclosure, a method for treating and preventing a skin disorder in a subject in need of such treatment, comprises administering to the subject a therapeutically effective amount of a composition comprising a neutralizing agent directed against the organism associated with the skin disorder, one or more synergistic agents selected from the group consisting of anti-acne actives, anti-microbial actives, antifungal actives, anti-inflammatory actives, exfoliating agents and mixtures thereof, and a pharmaceutically acceptable carrier. In one embodiment, the skin disorder is acne vulgaris. In some embodiments, the neutralizing agent is directed against the bacteria associated with acne vulgaris. In one embodiment, the neutralizing agent comprises an antibody against Propionibacterium Acnes. In some embodiments, the one or more synergistic agents comprise anti-acne actives. In other embodiments, the one or more synergistic agents comprise an exfoliating agent. In one example, the one or more synergistic agents comprise salicylic acid and/or salicylic derivatives.

Other aspects and variations of the compositions and methods summarized above are also contemplated and will be more fully understood when considered with respect to the following disclosure.

### DETAILED DESCRIPTION

The present disclosure provides compositions and methods for treating skin disorders, including acne vulgaris. In various embodiments, the present composition comprises a neutralizing agent directed against one or more organisms associated with the skin disorders and one or more synergistic agents.

As used herein, the term “organism” refers to any living biological entity, such as an animal, plant, fungus, or bacterium.

As used herein, the term “neutralizing agent” refers to any naturally occurring or synthetic substance, compound or molecule directed specifically against a target organism and/or a related organism from the same species, genus or family, which has the ability to destroy, inhibit the growth of the organism of interest, and/or neutralize the effects of the organism of interest. Thus, the neutralizing agent can include a nucleic acid specific to one or more regions of the target organism genome. The neutralizing agent can also include a peptide interacting specifically with one or more parts of the target organism. The neutralizing agent can also include an antibody directed against the whole target organism or a specific epitope on the target organism. The neutralizing agent can also encompass other molecules or substances that may specifically interact with the target organism via unknown mechanisms and thereby neutralize their effects.

As used herein, the term “antibody” and “immunoglobulin” refer to any full-length or fragmentary antigen binding protein produced either naturally or by recombinant means. Antibodies that may be used with the present compositions include polyclonal, monoclonal, recombinant single-chain antigen binding proteins, and other recombinant immunoglobulins (e.g. chimeric antibodies). The term “immunoglobulin” also refers to either a single immunoglobulin or a mixture of immunoglobulins. Examples of immunoglobulins include, but are not limited to IgA, IgD, IgE, IgG, IgM, IgY and fragments thereof (e.g. Fab, Fc, and F′(ab′)₂ fragments. Antibodies that may be used with the present compositions may be isolated from any species, including rabbits, mice, chickens, goats, or birds.

As used herein, the term “skin area affected by acne” refers to areas of the body displaying symptoms of acne. The term also encompasses areas in which organisms associated with acne are present.

As used herein, the term “synergistic agent” refers to any naturally occurring or synthetic compound, substance or molecule which has the ability to enhance the activity of the neutralizing agent against a target organism.

As used herein, the term “therapeutically effective amount” refers to an amount of the composition or compound which, depending on the selected mode, frequency and duration of administration, produces a desired effect. For topical administration, the term “therapeutically effective amount” refers to an amount that is suitable for use in contact with human skin and tissues without undue toxicity, irritation, incompatibility, instability, allergic response, and the like.

According to aspects of the present disclosure, there is provided a composition for the treatment of a skin disorder, wherein the composition comprises a neutralizing agent directed against the organism associated with the skin disorder and one or more synergistic agents. In one embodiment, the skin disorder is of bacterial origin. In one embodiment, the skin disorder is acne vulgaris. In several embodiments, the neutralizing agent is directed against one or more organisms associated with acne vulgaris. In some embodiments, the neutralizing agent is directed against Propionibacterium acnes (P. acnes). In alternative embodiments, the neutralizing agent is directed against P. granulosum. In yet alternative embodiments, the neutralizing agent is directed against other members of the Propionibacteria family, including but not limited to P. acidipropionici; P. australiense; P. avidum; P. cyclohexanicum; P. freudenreichii; P. jensenii; P. microaerophilum; P. propionicum; and P. thoenii. In some embodiments, the neutralizing agent comprises a mixture of molecules, compounds or substances directed against several members of the Propionibacteria family.

In some embodiments, the neutralizing agent comprises an antibody directed against one or more organisms associated with acne vulgaris. In alternative embodiments, the neutralizing agent comprises one or more nucleic acids which can specifically bind to specific sequences of the genetic material of the organisms of interest, and thereby inhibit their growth.

In several embodiments, the neutralizing agent comprises an immunoglobulin against P. acnes. In some embodiments, the neutralizing agent comprises a mixture of antibodies directed against several members of the Propionibacteria family. In some embodiments, the neutralizing agent comprises an immunoglobulin directed against different epitopes of a same bacterium.

In one embodiment, the neutralizing agent comprises an immunoglobulin directed against the whole bacteria of P. acnes. In one example, the immunoglobulin is an IgY directed against the whole bacteria of P. acnes. In alternative embodiments, the neutralizing agent comprises anti-P. acnes IgA, IgG, IgE, IgM, IgD or a mixture thereof. In yet alternative embodiments, the neutralizing agent comprises one or more immunoglobulins directed against one or more specific part of P. acnes.

The composition according to the present disclosure comprises a neutralizing agent and one or more synergistic agents. In some embodiments, the synergistic agent comprises one or more active ingredients from the group including anti-bacterial actives, anti-acne actives, anti-inflammatory actives, antifungal actives, keratolytic actives, oxidizing agents, anti-oxidants, exfoliating agents, and/or mixtures thereof.

In any embodiment of the present disclosure, the actives useful herein can be categorized by the benefit they provide or by their postulated mode of action. However, it is to be understood that the actives useful herein can in some instances provide more than one benefit or operate via more than one mode of action. Therefore, classifications herein are made only for convenience and are not intended to limit the actives to that particular application or applications listed.

Examples of useful anti-acne actives that may be used as synergistic agents in the present composition include keratolytic agents such as salicylic acid (o-hydroxybenzoic acid), derivatives of salicylic acid such as 5-octanoyl salicylic acid, and resorcinol; retinoids such as retinoic acid and its derivatives (e.g., cis and trans); sulfur-containing D and L amino acids and their derivatives and salts; antibiotics and antimicrobials such as benzoyl peroxide, octopirox, tetracycline, 2,4,4′-trichloro-2′-hydroxy diphenyl ether, 3,4,4′-trichlorobanilide, azelaic acid and its derivatives, phenoxyethanol, phenoxypropanol, phenoxisopropanol, ethyl acetate, clindamycin and meclocycline; sebostats such as flavonoids; and bile salts such as scymnol sulfate and its derivatives, deoxycholate, cholate, and/or mixtures thereof.

Examples of useful exfoliating agents which may be used as synergistic agents in the present composition includes lactic acid and derivatives, salicylic acid and derivatives, fruit enzymes, alpha hydroxyl acid(s), glycolic acid(s), surfactants, and/or mixtures thereof.

Examples of useful anti-inflammatory actives which may be used as synergistic agents in the present composition includes propionic acid derivatives; acetic acid derivatives; fenamic acid derivatives; biphenylcarboxylic acid derivatives; and oxicams; acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, microprofen, tioxaprofen, suprofen, alminoprofen, tiaprofenic acid, fluprofen, bucloxic acid, hydrocortisone, and/or mixtures thereof.

Examples of useful anti-microbial and anti-fungal actives that may be used as synergistic agents in the present composition includes beta.-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin.

In several embodiments, the synergistic agent of the present composition is selected from salicylic acid, benzoyl peroxide, 3-hydroxy benzoic acid, glycolic acid, lactic acid, 4-hydroxy benzoic acid, acetyl salicylic acid, 2-hydroxybutanoic acid, 2-hydroxypentanoic acid, 2-hydroxyhexanoic acid, phytic acid, N-acetyl-L-cysteine, lipoic acid, azelaic acid, arachidonic acid, benzoylperoxide, tetracycline, ibuprofen, naproxen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neocycin sulfate, and mixtures thereof.

In one embodiment, the synergistic agent of the present composition comprises salicylic acid and/or salicylic derivatives.

In another aspect, the present composition comprises a therapeutically effective amount of a neutralizing agent directed against the organism associated with the skin disorders and one or more synergistic agents. A therapeutically effective amount can be an amount sufficient to alleviate one or more symptoms of acne. The therapeutically effective amount can be determined empirically and depends on a number of factors, including but not limited to the severity of the acne symptoms and the route of administration.

In some embodiments, the present composition comprises a therapeutically effective amount of immunoglobulin directed against anti-P. acnes and salicylic acid and/or salicylic derivatives. In some embodiments, the therapeutically effective amount of immunoglobulin ranges from about 0.0001% (or 0.1 ppm) to about 1%, more preferably from about 0.0005% (0.5 ppm) to about 0.01% (10 ppm), and most preferably from about 0.001% (1 ppm) to about 0.005% (5 ppm). In one specific embodiment, the present composition comprises about 3 ppm anti-P. acnes IgY at 95% purity and one or more synergistic agents.

In some embodiments, the therapeutically effective amount of salicylic acid and/or salicylic derivatives ranges from about 0.1% to about 10%, more preferably from about 0.2% to about 5%, most preferably from about 0.5% to about 2%. Compositions comprising from about 0.5% to about 2% salicylic acid and/or derivatives are suitable for over the counter use. Compositions comprising higher than 2% salicylic acid and/or derivatives may require prescription from a physician.

In one specific embodiment, the present composition comprises about 3 ppm anti-P. acnes IgY at 95% purity and about 2% salicylic acid and/or salicylic derivatives.

The present composition may comprise one or more pharmaceutically acceptable carriers. As used herein, “pharmaceutically acceptable carrier” refers to any agents which do not cause an intolerable side effect and which allow the active ingredients in the present composition to retain their pharmacological activities. A pharmaceutically acceptable carrier includes excipients, emulsifiers, solubilizers, surfactants, buffers, preservatives, and/or other additives which may enhance stability, delivery, absorption, half-life, efficacy, pharmacokinetics, pharmacodynamics, reduce adverse side effect or provide other advantages for pharmaceutical use. The CTFA Cosmetic Ingredient Handbook, Second Edition, 1992, which is incorporated by reference herein in its entirety, describes a wide variety of cosmetic and pharmaceutical ingredients commonly used in the skin care industry, which are suitable for use in the compositions provided according to the present disclosure. Examples of these functional classes include: absorbents, abrasives, anti-acne agents, anticaking agents, antifoaming agents, antimicrobial agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticers, preservatives, propellants, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), sunscreen agents, ultraviolet light absorbers, and viscosity increasing agents (aqueous and nonaqueous). Examples of other functional classes of materials useful herein that are well known to one of ordinary skill in the art include emulsifiers, sequestrants, skin sensates, and the like.

Additional components cited in the CFTA Ingredient Handbook which are suitable for use in the compositions provided according to the present disclosure include: vitamins and derivatives thereof (e.g., vitamin C, Vitamin A (i.e. retinoic acid), Vitamin B3, retinol, esters of retinoic acid, esters of retinol, retinoids, pathenol, pathenol esters, tocopherol, tocopherol esters, and the like); oil or sebum control agents such as clays silicones and drug actives; sunscreening agents; other silicone material such as dimethiconol, dimethicone copolyol, and amodimethicone, and the like; polyethyleneglycols and polypropyleneglycols; polymers for aiding the film-forming properties and substantivity of the compositions (such as a copolymer of eicosene and vinyl pyrrolidone).

The choice of a suitable pharmaceutically acceptable carrier will depend on the exact nature of the particular formulation desired, e.g., whether the present composition is to be formulated into a liquid solution, a suspension, an ointment, a film or a gel. The choice of a suitable pharmaceutically acceptable carrier will also depend on the route of administration. Preferably, the carrier is formulated to be suitable for a chosen route of administration.

The present composition may be administered via topical, subcutaneous, or transdermal administration. In some embodiments, the present composition is administered topically.

In some embodiments, the present composition is prepared and packaged in a formulation suitable for topical administration. Topical formulations include gels, creams, ointments, salve, lotion, liquids, foam, and aerosol. Alternatively, the present composition may be formulated in a microcrystalline form, in a liposomal preparation or as a wipe. The present composition may be formulated to be used as a cleanser or a toner. The present composition may be formulated to be used on the whole surface of a target area or for spot treatment. Formulations suitable for a desired route of administration are within the skill of one in the art.

Thus, aspects of the present disclosure provide for a composition for topical treatment of skin disorders including acne vulgaris, the composition comprising a neutralizing agent, one or more synergistic agents selected from the group of anti-acne actives, anti-microbial actives, antifungal actives, anti-inflammatory actives, exfoliating agents and mixtures thereof; and a pharmaceutically acceptable carrier. In one embodiment, the neutralizing agent comprises an immunoglobulin directed against P. acnes and the synergistic agent comprises salicylic acid and/or derivatives in a pharmaceutically acceptable carrier. By way of examples, the composition may comprise between 0.1% and 50% (w/w) active ingredients, and 50% to 99.9% (w/w) pharmaceutical acceptable carrier.

In another aspect of the present disclosure, there is provided a method for the treatment of a skin disorder in a subject in need thereof, wherein the method comprises administering to the subject a therapeutically effective amount of a composition comprising a neutralizing agent directed against the organism associated with the skin disorder, one or more synergistic agents selected from the group consisting of anti-acne actives, anti-microbial actives, antifungal actives, anti-inflammatory actives, exfoliating agents and mixtures thereof; and a pharmaceutically acceptable carrier. In one embodiment, the administering is topical, whereby the treatment is applied to a skin area affected by acne. Compositions suitable for the present method are disclosed herein.

The present disclosure also provides for a method for alleviating acne-associated symptoms, the method comprises administering to a skin area affected by acne a therapeutically effective amount of a composition comprising a neutralizing agent directed against the organism associated with acne, one or more synergistic agents selected from the group consisting of anti-acne actives, anti-microbial actives, antifungal actives, anti-inflammatory actives, exfoliating agents and mixtures thereof; and a pharmaceutically acceptable carrier. Compositions suitable for alleviating acne-associated symptoms are disclosed herein.

The following examples are presented to set forth more clearly the subject matter of this disclosure without imposing any limits on the scope thereof.

Example 1 is an illustrative and non-limiting list of ingredients of a composition prepared according to aspects of the present disclosure.

| PHASE | Ingredients | % W/W |
|---|---|---|
| A | Water | 88.57 |
| | Disodium EDTA | 0.05 |
| B | Hydroxyethyl Acrylate/Sodium | 2.00 |
| | Acryloyldimethyl | |
| | Taurate Copolymer & Polyisobutene | |
| | & PEG-7 | |
| | Trimethylolpropane | |
| | Coconut Ether | |
| | Polyacrylate Crosspolymer-6 | 0.75 |
| C | Glycerin | 2.00 |
| | Methylpropanediol | 1.00 |
| | Propanediol | 3.00 |
| | Salicylic Acid | 2.00 |
| D | Water, Sodium Hydroxide | 0.00 |
| E | Phenoxyethanol, | 0.60 |
| | Methylisothiazolione | |
| | Anti-P. acnes IgY 1% solution | 0.03 |

Example 2 describes an illustrative and non-limiting procedure that can be used to prepare the composition presented in Example 1.

### Example 2

Step 1: Add water to a main vessel, and begin propeller agitation. Add the rest of the ingredients in phase A, and mix until fully dissolved.

Step 2: Begin adding ingredients in phase B to the main vessel. Add ingredients in the order given, and make sure each ingredient is fully hydrated and homogenous before adding the next ingredient.

Step 3: In a separate vessel, add ingredients in phase C. Mix these ingredients until the Salicylic acid is fully dissolved. Add this solution to the solution in the main vessel.

Step 4: Using 20% solution of sodium hydroxide, adjust the pH to between 3.8-4.5.

Step 5: Begin adding the ingredients in phase E to the main vessel. Ensure each ingredient is homogenous before adding the next ingredient.

### Example 3

In vitro experiments show the effectiveness of a composition comprising salicylic acid and anti-P. acnes immunoglobulin at reducing a specific microorganism population.

The microorganism of interest in the described experiments was Propionibacterium acnes. A stock suspension of P. acnes was prepared by inoculating a sample of P. acnes (ATCC #6919) in Reinforced Clostria broth at 36±1° C. for 5 days anaerobically. This stock suspension was divided into four equal batches. The first batch served as a no treatment control group. The second batch was treated with 2% salicylic acid. The third batch was treated with 3 ppm anti-P. acnes IgY at 95% purity. The fourth batch was treated with 2% salicylic acid and 3 ppm anti-P. acnes IgY at 95% purity. At time intervals 30 seconds, 1 minute, 5 minutes, 12 hours, and 24 hours, 1.0 ml from the inoculated control and test batches was taken and placed into 9.0 ml of neutralizing broth (1:10 dilution). Additional dilutions were made using neutralizing broth to achieve 1:100, 1:1000 and 1:10,000 dilutions. One milliliter from each dilution was plated into TSA+5% sheep blood. The plates were incubated at 36±1° C. for 5 days anaerobically. After the incubation period, all plates were counted to determine the number of microorganism at each time point.

The results of the experiments described in Example 3 are shown in Table 1:

TABLE 1

| | | | | | 2% SA + |
|---|---|---|---|---|---|
| Exposure Time | | Control | 2% SA | 3 ppm IgY | 3 ppm IgY |
| Initial | | 8.20E+05 | 8.20E+05 | 8.20E+05 | 8.20E+05 |
| 30 | sec. | 1.60E+06 | 7.20E+05 | 1.80E+06 | <10 |
| 1 | min. | 1.40E+06 | n/a | 2.20E+06 | <10 |
| 5 | min. | 9.40E+05 | n/a | 1.20E+06 | <10 |
| 12 | hours | 2.40E+06 | <10 | <10 | <10 |
| 24 | hours | 1.70E+06 | <10 | <10 | <10 |

Notes:
SA refers to salicylic acid, IgY refers to IgY directed against anti-P. acnes whole bacteria.

According to the obtained results, the composition comprising 2% salicylic acid and 3 ppm anti-P. acnes IgY reduced over 99% the concentration of P. acnes within the first 30 seconds, which remained low (at <10) for all remaining test times. In contrast, 2% salicylic acid alone had only a marginal effect of about 12% reduction and 3 ppm anti-P. acnes IgY alone did not seem to have any reduction effect during the first 30 seconds. Relative to the combination of 2% SA and 3 ppm anti-P. acnes IgY, both 2% salicylic acid and 3 ppm anti-P. acnes IgY when used alone reached a 99% kill rate only after 12 hours. These results show that the combination of 2% SA and 3 ppm anti-P. acnes IgY was much more effective in killing P. acnes than individual ingredients added alone.

Thus, the present disclosure provides for compositions and methods for treating skin disorders including acne vulgaris. In one specific embodiment, antibodies directed to one or more organisms associated with acne vulgaris in combination with one or more synergistic agents are used for treating acne. In the non-limiting examples provided, salicylic acid was provided as an exemplary synergistic agent. The present disclosure should not however be construed to be limited to the examples provided, since organisms associated with acne, other than P. acnes, P. propilosum, and the remaining members of the Propionibacterium family, which at present unknown, once discovered, may be used in accordance to the methods and compositions of the present disclosure.

Many alterations and modifications may be made by those having ordinary skill in the art, without departing from the spirit and scope of the disclosure. Therefore, it must be understood that the described embodiments have been set forth only for the purposes of examples, and that the embodiments should not be taken as limiting the scope of the following claims. The following claims are, therefore, to be read to include not only the combination of elements which are literally set forth, but all equivalent elements for performing substantially the same function in substantially the same way to obtain substantially the same result. The claims are thus to be understood to include those that have been described above, those that are conceptually equivalent, and those that incorporate the ideas of the disclosure.

What is claimed is:

1. A pharmaceutical composition for treating a skin disorder, the composition comprising:
a neutralizing agent directed against at least one organism associated with the skin disorder;
a synergistic agent selected from the group consisting of anti-acne actives, anti-microbial actives, antifungal actives, anti-inflammatory actives, exfoliating agents and mixtures thereof; and
a pharmaceutically acceptable carrier.

2. The pharmaceutical composition of claim 1, wherein the skin disorder is of bacterial origin, wherein the neutralizing agent is directed against at least one species of bacteria associated with the skin disorder, and wherein the synergistic agent is selected from the group consisting of anti-acne actives, anti-microbial actives, anti-inflammatory actives, exfoliating agents and mixtures thereof.

3. The pharmaceutical composition of claim 2, wherein the skin disorder of bacterial origin is acne vulgaris.

4. The pharmaceutical composition of claim 2, wherein the synergistic agent comprises salicylic acid.

5. The pharmaceutical composition of claim 2, wherein the neutralizing agent comprises an antibody directed against the at least one species of bacteria associated with the skin disorder.

6. The pharmaceutical composition of claim 5, wherein the antibody comprises an immunoglobulin directed against Propionibacterium acnes.

7. The pharmaceutical composition of claim 4, wherein the synergistic agent comprises from about 0.5% to about 2% salicylic acid.

8. The pharmaceutical composition of claim 6, wherein the neutralizing agent comprises from about 1 ppm to about 5 ppm immunoglobulin directed against P. acnes.

9. A method for treating a skin disorder in a subject in need of such treatment, comprising: administering to the subject a therapeutically effective amount of a composition comprising (a) a neutralizing agent directed against at least one organism associated with the skin disorder; (b) one or more synergistic agents selected from the group consisting of anti-acne actives, anti-microbial actives, antifungal actives, anti-inflammatory actives, exfoliating agents and mixtures thereof; and (c) a pharmaceutically acceptable carrier.

10. The method of claim 9, wherein the skin disorder is of bacterial origin.

11. The method of claim 10, wherein the skin disorder includes acne vulgaris.

12. The method of claim 9, wherein the one or more synergistic agents comprises salicylic acid.

13. The method of claim 10, wherein the neutralizing agent comprises an antibody directed against at least one species of bacteria associated with the skin disorder.

14. The method of claim 11, wherein the antibody comprises an immunoglobulin directed against Propionibacterium Acnes.

15. The method of claim 9, wherein the synergistic agent comprises from about 0.5% to about 2% salicylic acid.

16. The method of claim 14, wherein the neutralizing agent comprises from about 1 ppm to about 5 ppm immunoglobulin directed against P. acnes.

17. The method of claim 9, wherein the administering is topical.

18. The method of claim 9, wherein the administering is subcutaneous.

19. The method of claim 9, wherein the administering is transdermal.
